(19) United States
(12) Patent Application Publication
(10) Pub. No.: US 2015/0051559 A1
(43) Pub. Date: Feb. 19, 2015

(54) TRANSDERMAL PATCH FOR TREATMENT OF DEMENTIA OR ALZHEIMER TYPE DEMENTIA

(71) Applicant: SPARSHA PHARMA INTERNATIONAL PRIVATE LIMITED, Hyderabad (IN)

(72) Inventor: Noriyuki Kuzumaki, Hyderabad (IN)

(21) Appl. No.: 14/388,094

(22) PCT Filed: Mar. 18, 2013
(86) PCT No.: PCT/IN2013/000163

§ 371 Date: Sep. 25, 2014

(30) Foreign Application Priority Data
Apr. 5, 2012 (IN) 1383/CHE/2012

(51) Int. Cl. A61K 9/70; A61K 47/10; A61K 31/27

(52) U.S. Cl. CPC A61K 9/7053; A61K 31/27; A61K 47/10
USPC 604/307

(57) ABSTRACT

Disclosed is a transdermal patch for treatment of degenerative neurological disorders like dementia or Alzheimer type dementia. More particularly, the transdermal patch for treatment of neurodegenerative disorder comprises an adhesive monolayer which comprises Rivastigmine in free base form or its pharmaceutically acceptable salts, styrene block copolymer, and a tackifier, wherein total styrene units are in an amount of 4% w/w or more of said adhesive monolayer.

### FIELD OF INVENTION

The present invention relates to a transdermal patch for treatment of degenerative neurological disorders like dementia or Alzheimer type dementia.

### BACKGROUND

There are certain neurological disorders in which there will be progressive deterioration in cognitive function. Dementia is the one among them and it is a non-specific syndrome in which affected areas of brain function may be affected, such as memory, language, problem solving and attention. Dementia, unlike Alzheimer's, is not a disease in itself. When dementia appears the higher mental functions of the patient are involved initially. Eventually, in the later stages, the person may not know what day of the week, month or year it is, he may not know where he is, and might not be able to identify the people around him. This dementia is one major symptom associated with Alzheimer's, Parkinson's etc.

Certain drugs like Rivastigmine, Donepezil and Galantamine etc., are known in the prior art to be used in treatment of Alzheimer type dementia.

U.S. Pat. No. 4,948,807 discloses a compound N-ethyl, N-methyl-3-[1-(dimethylamino)ethyl]-N-methyl-phenyl-carbamate] with common name rivastigmine for the treatment of senile dementia, Alzheimer's disease and other neurodegenerative disorders.

Rivastigmine is available commercially in the market as oral capsule and solution dosage forms under the brand name EXELON®.

The oral administration of rivastigmine or other known anti-dementia medicines is generally followed or accomplished by nausea and vomiting. Since, a patient has to take these medicines for a long duration therefore, a strong need was felt for alternate route of administration of these anti-dementia drugs to overcome the associated side effects. Systemic administration of anti-dementia drugs has been found to be very effective alternative in lowering the above specified side effects like nausea and vomiting. In systemic administration the active drug diffuses through the skin in transdermal route.

In recent developments, transdermal patches comprising anti-dementia drugs have been very successful in treatment of these neurodegenerative disorders with less side effects and better patient compliance.

U.S. Pat. No. 5,602,176 discloses (S)-N-ethyl-3-[(1-dimethylamino) ethyl]-N-methyl-phenylcarbamate (rivastigmine) and a transdermal pharmaceutical composition comprising rivastigmine and non-swellable acrylate polymer (Durotack 280-2416).

U.S. Pat. No. 6,316,023 claims a transdermal device comprising a pharmaceutical composition containing 1 to 40 weight percent of rivastigmine in free base or acid addition salt, 0.01 to 0.5 weight percent of an antioxidant and a diluent or carrier wherein the antioxidant is preferably α-tocopherol or ascorbylpalmitate. The composition also comprises 10 to 30 weight percent of polymethacrylate or acid addition salt.

US 2010/0087768 relates to transdermal device containing a drug which is liquid at room temperature, selected from propargylamines and rivastigmine which may be partially volatile at process temperature during manufacture of the device. In this patent acrylic polymer pressure sensitive adhesive is used for formulation.

WO 2011076621 relates to adhesive monolith comprising a drug reservoir (reservoir) on a support (top layer) containing rivastigmine as drug and a polymer matrix and an adhesive layer present on the reservoir comprising a contact adhesive and a removable layer wherein the polymer matrix of the reservoir does not comprise either hydroxyl groups or carboxyl groups Rivastigmine patch is commercially available (Exelon® patch) in the market. However, this commercially available rivastigmine patch has complicated multilayer pressure-sensitive adhesive system consisting of acrylic polymer layer containing drug, antioxidant and silicone adhesive layer to control drug release. It contains four layers i.e. backing film followed by drug matrix (drug product in acrylic matrix), adhesive (silicon) matrix and release liner which will be removed at the time of use.

This multilayer system always increase the production cost and has a more complex manufacture than monolayer systems. The acrylics used in the above discussed prior arts usually have a disadvantage of poor adhesion to low-energy surfaces, such as polyethylene and polypropylene, as well as lower overall adhesion compared to rubber unless the adhesive is highly engineered.

### OBJECTS OF THE INVENTION

The primary object of the invention is to provide a transdermal patch for treatment of neurodegenerative disorder like dementia or Alzheimer type dementia.

Another object of the invention is to provide a controlled release transdermal patch of rivastigmine, which is cost effective, more stable and less complicated for large scale industrial production.

### BRIEF DESCRIPTION OF THE DRAWINGS

FIG. 1 is graphical representation of in vitro mouse skin permeation test result for examples 1, 3 and 5.

FIG. 2 is graphical representation of in vitro mouse skin permeation test result for examples 7 and 11.

FIG. 3 is graphical representation of in vitro human skin permeation Test result for examples 1 and 7.

### SUMMARY OF THE INVENTION

Accordingly, the present invention provides a transdermal patch of rivastigmine for treatment of neurodegenerative disorder like dementia or Alzheimer type dementia by controlled systemic administration of rivastigmine to the subject under treatment.

In one embodiment of the invention, the transdermal patch comprises a backing film, an adhesive monolayer containing rivastigmine in free base form or its pharmaceutically acceptable salts and release liner.

In one preferred embodiment of the invention, the transdermal patch comprises an adhesive monolayer which contains rivastigmine, styrene block copolymer, a tackifier, and an anti-oxidant, wherein total polystyrene units are in an amount of 4% w/w or more of said adhesive monolayer.

### DETAILED DESCRIPTION

Detailed embodiments of the present invention are disclosed herein with reference to the examples and drawings. However, it is to be understood that the disclosed embodiments are merely exemplary of the invention, which can be embodied in various forms. Therefore, specific structural and functional details disclosed herein are not to be interpreted as limiting, but merely as a basis for the claims and as a representative basis for teaching one skilled in the art to variously employ the present invention in virtually any appropriately detailed structure. Further, the terms and phrases used herein are not intended to be limiting but rather to provide an understandable description of the invention.

The term “controlled release” as used herein refers to the drug release. Typically, the controlled release refers to the release of the drug at a controlled rate over an extended period of time longer in duration compared to the conventional release.

In the preferred embodiment, rivastigmine may be in the form of free base or its pharmaceutically available salts in the adhesive monolayer.

In one embodiment of the invention, the amount of rivastigmine in free base form is preferably 10-25%, more preferably 15-21% w/w of the adhesive monolayer.

It was found that styrene block copolymer is suitable as a synthetic rubber polymer in preparing the transdermal patch with rivastigmine as active anti-dementia drug. In the preferred embodiment, styrene block copolymer is at least one selected from the group consisting of styrene-butadiene-styrene, Styrene-ethylene/butylene-styrene, Styrene-ethylene/propylene, Styrene-isoprene-styrene. In more preferred embodiment, Styrene-isoprene-styrene is used.

In one embodiment of the invention, the amount of styrene block copolymer is preferably 10-35% w/w, more preferably 15-30% w/w, still more preferably 20-30% w/w of the adhesive monolayer.

In above monolayer, total amount of polystyrene unit may be preferably 4-6% w/w, more preferably 4.5-5.5% w/w, of said adhesive monolayer. Such amount of polystyrene content is advantageous for the good physicochemical property of softness (hardness), drug holding without having cold flow, and enable higher adhesiveness on skin.

Tackifiers are chemical compounds used in formulating adhesives to increase the tack, the stickiness of the surface of the adhesive. Tackifiers are usually resins (e.g. rosins and their derivates, terpenes and modified terpenes, aliphatic, cycloaliphatic and aromatic resins (C5 aliphatic resins, C9 aromatic resins, and C5/C9 aliphatic/aromatic resins), hydrogenated hydrocarbon resins, and their mixtures, terpene-phenol resins (TPR, used often with ethylene-vinyl acetate adhesives)).

Many pressure sensitive adhesives are a blend of styrene block copolymer and a tackifier resin. Some acrylic adhesives also include an additional tackifier. Rosin esters impart excellent specific adhesion to a wide range of substrates due to their polarity and polymer compatibility. Their low molecular weight and narrow molecular weight distribution, combined with their cycloaliphatic—aromatic structure, make rosin esters the most broadly compatible of all adhesive tackifiers. They are used in a wide range of polymers including high and low vinyl acetate EVA, acrylics, polyurethanes.

Further, the amount of the tackifier is preferably 30-70% w/w, more preferably 45-70% w/w, still more preferably 54-65% w/w of the monolayer. The ratio of total tackifier: styrene block copolymer is preferably 2:1 to 3:1, more preferably 2.1:1 to 2.6:1.

In one specific embodiment of the invention, the tackifier in the monolayer comprises rosin ester and petroleum resin. Preferred example of rosin ester and petroleum resin include rosins and their derivatives, terpenes and modified terpenes, aliphatic, cycloaliphatic and aromatic resins, hydrogenated hydrocarbon resins and their mixtures and terpene-phenol resins. The ratio of petroleum resin: rosin ester is preferably 1:1 to 3:1, more preferably 1.9:1 to 3:1, still more preferably 1.9:1 to 2.7:1.

Further, anti-oxidant is preferably a phenolic anti-oxidant, more preferably butylatedhydroxytoluene.

The amount of the anti-oxidant is in preferably 0.5% to 1.2% w/w of the monolayer.

The transdermal patch of the present invention may include a simple three layer laminate structure of an outer backing film, an adhesive monolayer and a release liner wherein said monolayer maybe a matrix structure as mentioned above.

The backing film is, without being limited to, selected from polyester film, polyester/polyethylene lamination film, or polyester/ethylene vinyl acetate lamination film.

The release liner film is, without being limited to, selected from silicon surface coated polyester film, fluoropolymer coated polyester, or fluoropolymer coated polypropylene. This release liner is removed at the time of usage.

The backing layer and the adjacent adhesive monolayer may have strong cohesive force between them which keeps the backing layer and matrix layer intact with each other when patch is applied on the body of the substrate whereas, the monolayer and the release liner may have weak cohesive force between them therefore, release liner can be easily removed before applying the patch on the body. Removal of release liner exposes the monolayer of the patch for application on the body wherein rivastigmine is systemically administered into the body.

The transdermal patch of the invention may provide a device for control release of rivastigmine for treatment of dementia or Alzheimer type dementia.

In another embodiment, the present invention also provides a process for preparing a pharmaceutical composition for use as the adhesive monolayer of the present invention wherein the process comprises:

a). dissolving styrene block copolymer in an organic solvent, preferably toluene;

b). adding tackifier such as Rosin ester and Petroleum resin and obtaining a clear semisolid mixture;

c). optionally adding other required excipients such as polybutene into the above mixture and thoroughly mixing the mixture contents;

d). adding rivastigmine into the mixture and mixing;

e). obtaining the pharmaceutical composition for use as the adhesive monolayer.

The process may further involves uniformly coating viscous solution on the silicone coated polyester film and drying in oven to remove solvent and it was laminated by polyester backing film, and cut by punching for required patch size. The transdermal patch of the invention can be easily produced and has good stability properties and sufficient stickiness for long lasting effect for once a daily application patch. The transdermal patch is also useful to simplify the manufacturing process and decreases the cost of production.

According to another embodiment, the present invention provides a method for treating neurodegenerative disorder comprising plastering on the skin of a living body with the transdermal patch of the present invention.

The amount of rivastigmine may be appropriately determined by those skilled in the art depending on the kinds of symptoms of patients, dosage periods, the sizes of preparations, and the like.

Also, the application period is preferably one day or 24 hours.

Further, the living body described above includes, for example, rabbit, dog, or human, preferably human.

### EXAMPLES

The invention is further explained with the help of following examples. However, given examples are only for understanding the invention and not for limiting the scope of the claims. In the example, “percentage” wherever used would mean “weight percentage”, unless otherwise specified.

Preparation of Transdermal Patch Formulation:

Total 13 different formulations of pharmaceutical composition with variation in the ingredients and concentrations of the ingredients were prepared as shown in following table 1. Below given general process was used for preparation of all 13 formulations. After preparation, all 13 formulations were evaluated for different parameters described herein after and the best formulation was identified with the combination and concentration of ingredients giving the best desired result.

Preparation of Transdermal Patch with Synthetic Rubber:

Synthetic rubber was dissolved in an organic solvent preferably toluene, tackfier materials such as Rosin ester and Petroleum resin were added and clear semisolid mixture was obtained. Polybutene was added as required as shown in the table-1. Other required materials as shown in table-1 were added and mixed. Rivastigmine base was added and well mixed. The viscous solution was uniformly coated on the silicone coated polyester film and dried in an oven to remove solvent, and it was laminated by polyester backing film, and cut by punching for required patch size.

Preparation of Transdermal Patch with Acrylic and Silicone Polymers:

Pre dissolved polymer solution in ethylacetate, hexane or other solvent was mixed with required materials, and followed by rivastigmine base and well mixed. The viscous solution was uniformly coated on the silicone coated polyester film and dried in oven to remove solvent, and it was laminated by polyester backing film, and cut by punching for required patch size.

TABLE 1
(Number: weight percentage in adhesive)

| | Ex- | Ex- | Ex- | Ex- | Ex- | Ex- | Ex- | Ex- | Ex- | Ex- | Ex- | Ex- | Ex- |
|---|---|---|---|---|---|---|---|---|---|---|---|---|---|
| INGREDIENTS | 1 | 2 | 3 | 4 | 5 | 6 | 7 | 8 | 9 | 10 | 11 | 12 | 13 |
| Rivastigmine base | 20.1 | 20.0 | 19.8 | 20.0 | 19.9 | 10.2 | 15.0 | 10.0 | 19.8 | 19.9 | 15.0 | 15.0 | 18.0 |
| Butylatedhydroxy | 0.5 | 0.5 | 0.5 | 0.5 | 0.5 | 0.5 | 0.8 | 0.8 | 0.6 | 0.6 | 0.8 | 0.8 | 1.0 |
| toluene | | | | | | | | | | | | | |
| Styrene-Isoprene- | 25.0 | — | — | — | — | — | 25.0 | 25.0 | 14.9 | 15.1 | — | — | — |
| Styrene copolymer | | | | | | | | | | | | | |
| (Polystyrene 22%) | | | | | | | | | | | | | |
| Styrene-Isoprene- | — | — | — | — | — | — | — | — | — | — | 25.0 | — | 25.0 |
| Styrene copolymer | | | | | | | | | | | | | |
| (Polystyrene 20%) | | | | | | | | | | | | | |
| Styrene-Isoprene- | — | — | — | — | — | — | — | — | — | — | — | 25.0 | — |
| Styrene copolymer | | | | | | | | | | | | | |
| (Polystyrene 15%) | | | | | | | | | | | | | |
| Rosin ester | 15.0 | — | — | — | — | — | 20.0 | 25.0 | — | 9.9 | 20.0 | 20.0 | 17.0 |
| Petroleum resin | 39.4 | — | — | — | — | — | 39.2 | 39.2 | 49.6 | 40.1 | 39.2 | 39.2 | 39.0 |
| Polybutene | — | — | — | — | — | — | — | — | 15.2 | 14.8 | — | — | — |
| Acrylates | — | — | 40.1 | — | — | — | — | — | — | — | — | — | — |
| Copolymer | | | | | | | | | | | | | |
| Durotak-2353 | | | | | | | | | | | | | |
| Acrylates | — | 79.5 | 39.5 | — | — | — | — | — | — | — | — | — | — |
| Copolymer | | | | | | | | | | | | | |
| Durotak-2510 | | | | | | | | | | | | | |
| Acrylates | — | — | — | — | 39.8 | — | — | — | — | — | — | — | — |
| Copolymer | | | | | | | | | | | | | |
| Cytec-9083 | | | | | | | | | | | | | |
| Acrylates | — | — | — | 79.5 | 39.8 | — | — | — | — | — | — | — | — |
| Copolymer | | | | | | | | | | | | | |
| Cytec-7883 | | | | | | | | | | | | | |
| Silicone | — | — | — | — | — | 89.3 | — | — | — | — | — | — | — |
| Adhesive | | | | | | | | | | | | | |
| Dowcorning- | | | | | | | | | | | | | |
| 4602 | | | | | | | | | | | | | |

Evaluation

Physical Observation of Adhesive

The physical property of adhesive was evaluated by thumb tack feeling. The evaluation score was indicated as below in table-2.

TABLE 2

| Score | |
|---|---|
| A | Excellent adhesion, no pithy nature, no remaining adhesive on |
| | thumb |
| B | Good adhesion, minor pithy nature, minor remaining adhesive on |
| | thumb |
| C | Fair adhesion, pithy nature, the adhesive remains on thumb |
| D | Poor adhesion, pithy nature, majority of adhesive remains on |
| | thumb |

Peel Test

The test sample was cut 1 cm width×2.5 cm long and placed on stainless steel plate and peeled with the angle of 180° Cat 300 mm/min speed. The peeling force was measured as adhesive test.

In Vitro Mouse Skin Permeation Test

The nude mouse (7-11 weeks old) skin was taken out surgically, and a 3 cm² of circled shape test sample was attached to the stratum corneum side of nude mouse skin and set on to Franz diffusion cells. The receiver compartment was filled with phosphate buffered saline, pH=7.4, at 32° C., and the receiver solution was taken at each predetermined sampling time point and measured by HPLC. The drug amount permeated was calculated as per the chromatographic peak area.

In Vitro Human Skin Permeation Test

Human cadaver skin was used to evaluate drug permeability of Example-1 and Example-2. A 5 cm² of circled shape test sample was attached to the homey side of human cadaver skin, and set on to flow-through type diffusion cells. The receiver compartment was filled with phosphate buffered saline, pH=7.4, at 32° C., and the receiver solution was taken at each predetermined sampling time point, and measured by HPLC. The drug amount permeated was calculated as per the chromatographic peak area.

FIG. 1 in the accompanying drawings illustrates a graphical representation of in vitro mouse skin permeation test result for examples 1, 3 and 5.

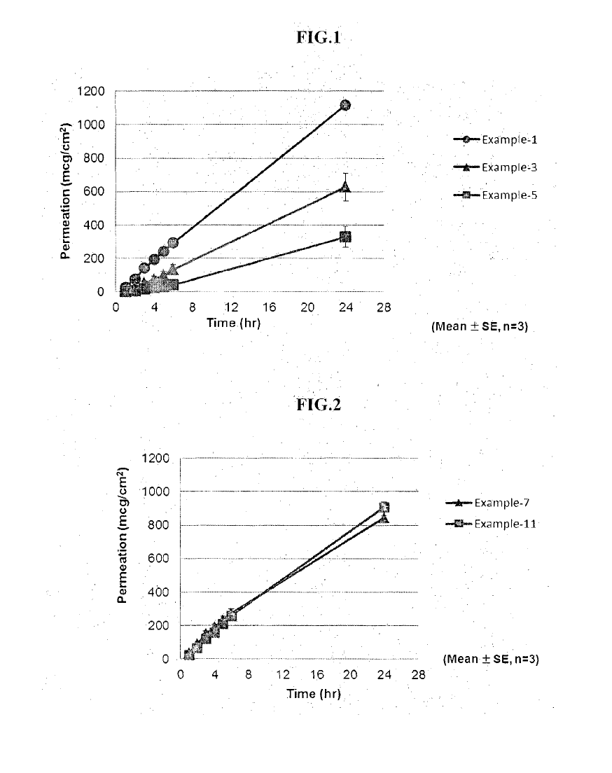

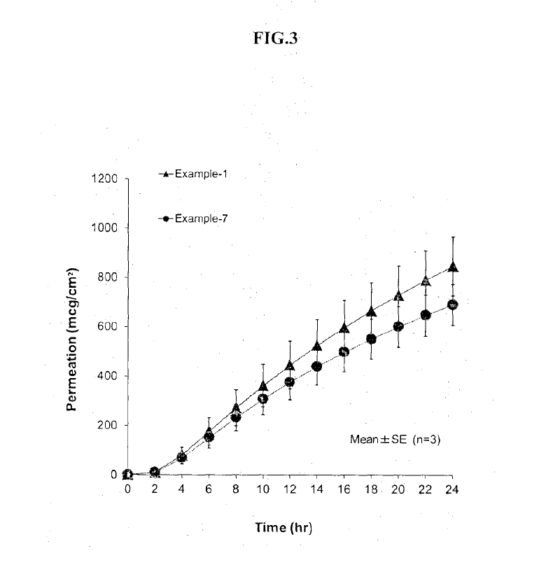

FIG. 2 in the accompanying drawings illustrates graphical representation of in vitro mouse skin permeation test result for examples 7 and 11.

Whereas, FIG. 3 of the accompanying drawings represents in vitro human skin permeation Test result for examples 1 and 7.

The observed results were summarized in table-3 below.

TABLE 3

| | | | | Ratio of | | | | | |
|---|---|---|---|---|---|---|---|---|---|
| | | Styrene- | | Tackifier/ | Ratio of | Total | | | |
| | | Isoprene- | | Styrene- | Petroleum | Polystyrene | | | |
| | | Styrene | | Isoprene- | resin/ | units | Physical | Peel | |
| | Drug | copolymer | Tackifier | Styrene | Rosin | content | Observation | Test | Skin |
| | (%) | (%) | (%) | copolymer | ester | (%) | Score | (kg/cm) | permeation |
| Ex-1 | 20.1 | 25.0 | 54.4 | 2.18 | 2.63 | 5.5 | A | 0.86 | A |
| Ex-2 | 20.0 | — | — | — | — | — | C | 1.03 | — |
| Ex-3 | 19.8 | — | — | — | — | — | B | 1.01 | B |
| Ex-4 | 20.0 | — | — | — | — | — | C | — | — |
| Ex-5 | 19.9 | — | — | — | — | — | B | 0.67 | C |
| Ex-6 | 10.2 | — | — | — | — | — | D | — | — |
| Ex-7 | 15.0 | 25.0 | 59.2 | 2.37 | 1.96 | 5.5 | A | 0.81 | A |
| Ex-8 | 10.0 | 25.0 | 64.2 | 2.57 | 1.96 | 5.5 | A | 1.63 | — |
| Ex-9 | 19.8 | 14.9 | 49.6 | 3.31 | — | 3.3 | C | — | — |
| Ex-10 | 19.9 | 15.1 | 50.0 | 3.33 | 4.05 | 3.3 | C | — | — |
| Ex-11 | 15.0 | 25.0 | 59.2 | 2.37 | 1.96 | 5.0 | A | 1.45 | A |
| Ex-12 | 15.0 | 25.0 | 59.2 | 2.37 | 1.96 | 3.75 | C | — | — |
| Ex-13 | 18.0 | 25.0 | 56.0 | 2.24 | 2.29 | 5.0 | A | 1.04 | — |

Observation

From the results, Example-1, 7, 8, 11 and 13 showed good score of physical observation, peel force value and higher skin permeability.

The composition of Example 1, 7, 8,11 and 13 is further described herein below in table-4.

TABLE 4

| | Ex- | Ex- | Ex- | Ex- | Ex- | Observed |
|---|---|---|---|---|---|---|
| | 1 | 7 | 8 | 11 | 13 | range |
| Drug (Rivastigmine | 20.1 | 15.0 | 10.0 | 15.0 | 18.0 | 15-20.1% |
| base) | | | | | | w/w |
| Anti-oxidant | 0.5 | 0.8 | 0.8 | 0.8 | 1.0 | 0.5-1.0% |
| | | | | | | w/w |
| Styrene-isoprene- | 25.0 | 25.0 | 25. | 25.0 | 25.0 | — |
| styrene | | | | | | |
| Total tackifier | 54.4 | 59.2 | 64.2 | 59.2 | 56.0 | 54.4-64.2% |
| (Rosin ester + | | | | | | w/w |
| Petroleum resin) | | | | | | |
| Ratio of total | 2.18 | 2.37 | 2.57 | 2.37 | 2.24 | 2.18-2.57 |
| tackifier/Styrene- | | | | | | |
| isoprene-styrene | | | | | | |
| Ratio of Petroleum | 2.63 | 1.96 | 1.96 | 1.96 | 2.29 | 1.96-2.63 |
| resin/Rosin ester | | | | | | |
| Total polystyrene | 5.5 | 5.5 | 5.5 | 5.0 | 5.0 | 5.0-5.5% |
| units content (%) | | | | | | |

1. A transdermal patch for treatment of neurodegenerative disorder of dementia or Alzheimer type dementia, comprising a backing film, an adhesive monolayer containing 10-25% of Rivastigmine in free base form or its pharmaceutically acceptable salts and a release liner, the adhesive monolayer further comprising styrene block co⁻polymer and tackifiers, wherein total polystyrene units are in an amount of 4%-6% w/w of said adhesive monolayer.

2-3. (canceled)

4. The transdermal patch as claimed in claim 1, wherein said styrene block copolymer is in an amount of 10-35% w/w of said adhesive monolayer.

5. (canceled)

6. The transdermal patch as claimed in claim 4, wherein said styrene block copolymer is selected from the group consisting of styrene-butadiene-styrene, Styrene-ethylene/butylene-styrene, Styrene-ethylene/propylene, and Styrene-isoprene-styrene.

7. The transdermal patch as claimed in claim 1, wherein said tackifier is a combination of rosin ester and petroleum resin, wherein the ratio of petroleum resin: rosin ester is 1:1 to 3:1.

8. (canceled)

9. The transdermal patch as claimed in claim 1, wherein said tackifier is in an amount of 30-70% w/w, of said adhesive monolayer.

10. The transdermal patch as claimed in claim 7, wherein the ratio of total tackifier: styrene block copolymer is 2:1 to 3:1.

11-14. (canceled)

15. The transdermal patch as claimed in claim 1, wherein said backing film is selected from polyester film, polyester/polyethylene lamination film, or polyester/ethylene vinyl acetate lamination film.

16. The transdermal patch as claimed in claim 1, further comprising a removable release liner selected from silicon surface coated polyester film, fluoropolymer coated polyester and fluoropolymer coated polypropylene.

17. (canceled)

18. The transdermal patch as claimed in claim 6, wherein the styrene block copolymer is Styrene-isoprene-styrene.

19. The transdermal patch as claimed in claim 9, wherein the tackifier is in an amount of 45-70% w/w of said adhesive monolayer.

20. The transdermal patch as claimed in claim 1, which further comprises an anti-oxidant.

21. The transdermal patch as claimed in claim 20, wherein said anti-oxidant is a phenolic anti-oxidant in an amount of 0.5% to 1.2% w/w of said adhesive monolayer.

22. The transdermal patch as claimed in claim 21, wherein said anti-oxidant is butylatedhydroxytoluene.
